(19) United States
(12) Patent Application Publication
(10) Pub. No.: US 2014/0302022 A1
(43) Pub. Date: Oct. 9, 2014

(54) 2-CARBOXAMIDE CLYCLOAMINO UREA DERIVATIVES FOR USE IN TREATING VEGF-DEPENDENT DISEASES

(71) Applicant: Christian Rene Schnell, Hegenhein (FR)

(72) Inventor: Christian Rene Schnell, Hegenhein (FR)

(73) Assignee: NOVARTIS AG, Basel (CH)

(21) Appl. No.: 14/354,365

(22) PCT Filed: Oct. 31, 2012
(86) PCT No.: PCT/EP2012/071614

§ 371 Date: Apr. 25, 2014

Related U.S. Application Data
(60) Provisional application No. 61/554,606, filed on Nov. 2, 2011.

(51) Int. Cl. C07D 417/14; A61K 45/06; A61K 31/4439

(52) U.S. Cl. CPC C07D 417/14; A61K 31/4439; A61K 45/06
USPC 424/133.1; 514/342; 514/248; 514/211.08

(57) ABSTRACT

The invention relates to the use of compounds of formula (I)

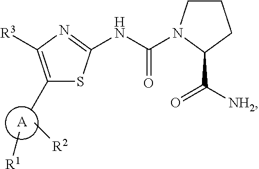

in the treatment of warm-blooded animal target of VEGF-driven angiogenic diseases, methods of use of said compounds in the treatment of said diseases in a warm-blooded animal, especially a human, pharmaceutical compositions comprising said compounds for the treatment of said diseases and said compounds for use in the treatment of said diseases.

The present invention relates to the use of specific 2-carboxamide cycloamino urea derivative compounds of formula (I), as described herein, in the treatment of VEGF-dependent diseases or for the manufacture of a pharmaceutical composition for use in the treatment of said diseases, methods of use of specific 2-carboxamide cycloamino urea derivative compounds in the treatment of said diseases in a warm-blooded animal, especially a human, pharmaceutical compositions comprising specific 2-carboxamide cycloamino urea derivative compounds for the treatment of said diseases and specific 2-carboxamide cycloamino urea derivative compounds for use in the treatment of said diseases.

The specific 2-carboxamide cycloamino urea derivative compounds of formula (I) show a strong selectivity for the phosphatidylinositol 3-kinase (PI3-kinase or PI3K) alpha subtype as compared to the beta, delta or gamma subtypes. It has been found that specific 2-carboxamide cycloamino urea derivative compounds, which have been described in WO2010/029082 to modulate the biological activity of PI3-kinase, are able to block the biological effects associated with the activation of VEGF receptors by their cognate ligands. Said compounds are thus useful for the treatment of VEGF-driven angiogenic diseases.

Syndromes with an established or potential molecular link to the VEGFR/VEGF axis are, for instance, described in “P. Carmeliet and R K Jain; Angiogenesis in cancer and other diseases, Nature 2000; 407: 249-257” and in S M Weiss and D A Cheresh; Pathophysiological consequences of VEGF-induced vascular permeability Nature 2005, 437:4697-50” which all are, including the references cited therein, hereby incorporated into the present application by reference, and are as follows:

Rheumatoid arthritis

Synovitis

Bone and cartilage destruction

Osteomyelitis

Pannus Growth

Osteophyte formation

Hepatitis

Pneumonia

Glomerulonephritis

Asthma

Nasal polyps

Transplantation

Liver generation

Retinopathy of prematurity

Age macular degeneration

Diabetic retinopathy

Chroidal and other intraocular disorders

Leukomalacia

Thyroiditis

Thyroid enlargement

Lympopholiferative disorders

Karposi's sarcoma

Haematologic malignacies (e.g., haemangiomas)

Obesity

Spinal cord injury

Acutemyocardial infarction

Pulmonary, cerebral and retinal oedema

or further any combinations thereof.

Current anti-angiogenic therapies aim to target either the binding of ligands (by competition with an antagonist or by trapping of the endogenous ligand or by expression of a soluble form of the receptor) on their cognate receptors expressed at the surface of endothelial cells composing the blood vessels (e.g. VEGF binding on VEGFR1, 2 and 3); or by impeding on the activation of the receptors by using small molecular mass inhibitors that block the kinase activity of the tyrosine kinase receptor(s) (e.g. blockade of VEGFR1, 2 or 3 activation). Other strategies aiming at upregulating endogenous and natural inhibitor of VEGF induced pathway in endothelial cells, or at attacking the already existing vasculature with a VEGF-toxin conjugate have already been described. PI3K inhibitors exert their anti-angiogenic properties by blocking the propagation of VEGF induced signal when bound to VEGFR1, 2 or 3. The PI3K/Akt pathway is an important VEGFR downstream effector as it is required for survival and proliferation of endothelial cells in vitro and in vivo (H P Gerber et al, Vascular Endothelial Growth Factor Regulates Endothelial Cell Survival through the Phosphatidylinositol 3′-Kinase/Akt Signal Transduction Pathway. J Biol Chem 1998; 273(46):30336-30343; Y Fujio Y, and K Walsh. Akt Mediates Cytoprotection of Endothelial Cells by Vascular Endothelial Growth Factor in an Anchorage-dependent Manner. J Biol Chem 1999; 274(23):16349-16354; L E Benjamin, and E Keshet E. Conditional switching of vascular endothelial growth factor (VEGF) expression in tumors: Induction of endothelial cell shedding and regression of hemangioblastoma-like vessels by VEGF withdrawal. PNAS 1997; 94(16):8761-8766; T L Phung et al. Pathological angiogenesis is induced by sustained Akt signaling and inhibited by rapamycin. Cancer Cell 2006; 10(2)159-170). PI3K inhibitors have been shown to abrogate VEGF induced proliferation and survival (“V Dayanir et al. Identification of Tyrosine Residues in Vascular Endothelial Growth Factor Receptor-2/FLK-1 Involved in Activation of Phosphatidylinositol 3-Kinase and Cell Proliferation. J Biol Chem 2001; 276(21):17686-17692.”), hence PI3K pathway interception is believed to have major effects on dysregulated vascular function (A K Olsson et al, Nature Review Molecular Cellular Biology, 2006; Vol 7, 359-371).

Specific 2-carboxamide cycloamino urea derivative compounds which are suitable for the present invention, their preparation and suitable pharmaceutical formulations containing the same are described in WO 2010/029082 and include compounds of formula (I)

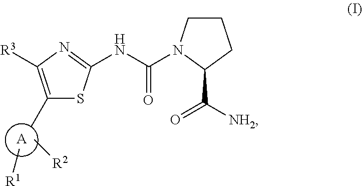

wherein
A represents a heteroaryl selected from the group consisting of:

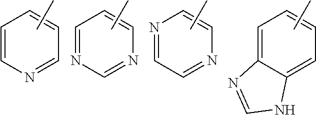

R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkylamino, di(C₁-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide).

The radicals and symbols as used in the definition of a compound of formula (I) have the meanings as disclosed in WO 2010/029082 which is hereby incorporated by reference in its entirety.

A preferred compound of formula (I) for the present invention is a compound which is specifically described in WO2010/029082. A very preferred compound of the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A) or a pharmaceutically acceptable salt thereof. The synthesis of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is described in WO2010/029082 as Example 15.

The compounds of formula (I) may be administered to a warm-blooded animal in need thereof in free base form or as a pharmaceutically acceptable salt. “Salts” (which, what is meant by “or salts thereof” or “or a salt thereof”), can be present alone or in mixture with free compound, e.g. the compound of the formula (I), and are preferably pharmaceutically acceptable salts. Such salts of the compounds of formula (I) are formed, for example, as acid addition salts, preferably with organic or inorganic acids, from compounds of formula (I) with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula (I) are preferably pharmaceutically acceptable salts; suitable counter-ions forming pharmaceutically acceptable salts are known in the field. “Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

According to the present invention the treatment of:

Rheumatoid arthritis

Synovitis

Bone and cartilage destruction

Osteomyelitis

Pannus Growth

Osteophyte formation

Hepatitis

Pneumonia

Glomerulonephritis

Asthma

Nasal polyps

Transplantation

Liver generation

Retinopathy of prematurity

Age macular degeneration

Diabetic retinopathy

Chroidal and other intraocular disorders

Leukomalacia

Thyroiditis

Thyroid enlargement

Lympopholiferative disorders

Karposi's sarcoma

Haematologic malignacies (e.g., haemangiomas)

Obesity

Spinal cord injury

Acutemyocardial infarction

Pulmonary, cerebral and retinal oedema

Or any further combination thereof.

with compounds of formula (I) are especially preferred:

In particular, the present invention relates to a method of treating a VEGF-driven angiogenic disease comprising administering a therapeutically effective amount of a specific 2-carboxamide cycloamino urea derivative compound of formula (I), especially preferred (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A), or a pharmaceutically acceptable salt thereof to a warm-blooded animal, particularly a human, in need thereof. “Therapeutically effective” preferably relates to an amount that is therapeutically or in a broader sense also prophylactically effective against the progression of a disease.

The present invention further relates to a compound of formula (I), especially preferred (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A), or a pharmaceutically acceptable salt for use in the treatment of a VEGF-driven angiogenic disease or malignancy or a disease that has acquired resistance to agents that target VEGF and/or VEGFR family members.

The present invention further relates to the use of a specific 2-carboxamide cycloamino urea derivative compound of formula (I), especially preferred (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A), or a pharmaceutically acceptable salt thereof for the manufacture of a pharmaceutical composition or medicament for the treatment of a VEGF-driven angiogenic disease or malignancy or a disease that has acquired resistance to agents that target VEGF and/or VEGFR family members. The terms “pharmaceutical preparation” or “pharmaceutical composition” refer to a mixture or solution containing at least one therapeutic compound to be administered to a warm-blooded animal, preferably a human, in order to prevent, treat or control a particular disease or condition affecting the warm-blooded animal.

The resistance to the treatment with a VEGF and/or VEGFR modulator can be acquired during treatment with said VEGF and/or VEGFR modulator by different mechanisms

In particular, the present invention relates to the treatment of a disease or malignancy that is dependent on VEGF or has acquired resistance during treatment with a modulator of the VEGF/VEGFR axis, with compounds of formula (I), especially preferred (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A), or a pharmaceutically acceptable salt thereof. Possible agents that target the VEGF/VEGFR axis are, for instance Bevacizumab, Ranibizumab, AVE0005, HuMV833, 2C3, CBO-P11, Sutent, Sorafenib, Vatalanib, Zactima, Midostaurin, Angiozyme, AG-013736, Lestautinib, CP-547,632, CEP-7055, KRN633, NVP-AEE788, IMC-1211, ZK260253, Semaxanib, E-7107, AS-3, Cand5 and PTC-299.

A compound of the formula (I) may also be used for the treatment of VEGF-driven angiogenic diseases in combination with other active compounds for instance the combination partners as disclosed in WO2010/029082, more preferred VEGF or VEGFR targeting agents such as, and without limitation instance anti-VEGF Bevacizumab, anti-VEGF, Ranibizumab AVE0005, anti-VEGF HuMV833, anti-VEGF 2C3, anti-VEGF CBO-P11, Sutent, Sorafenib, Vatalanib, Zactima, Midostaurin, Angiozyme, AG-013736, Lestautinib, CP-547,632, CEP-7055, KRN633, NVP-AEE788, IMC-1211, ZK260253, Semaxanib, E-7107, AS-3, Cand5 and PTC-299; and the HSP90 inhibitors CNF1010, CNF2024, tanespimycinm, alvespimycin, IP1504, SNX5422 and NVP-AUY922.

In one embodiment, the compound of formula (I) is used for the treatment of VEGF-driven angiogenic disease in combination with other active compounds, for instance the VEGF or VEGFR targeting agents such as, and without limitation instance anti-VEGF, Ranibizumab AVE0005, anti-VEGF HuMV833, anti-VEGF 2C3, anti-VEGF CBO-P11, Sutent, Sorafenib, Vatalanib, Zactima, Midostaurin, Angiozyme, AG-013736, Lestautinib, CP-547,632, CEP-7055, KRN633, NVP-AEE788, IMC-1211, ZK260253, Semaxanib, E-7107, AS-3, Cand5 and PTC-299; and the HSP90 inhibitors CNF1010, CNF2024, tanespimycinm, alvespimycin, IPI504, SNX5422 and NVP-AUY922.

The structure of the above active compounds identified by code nos., generic or trade names may be taken from the actual edition of the standard compendium “The Merck Index” or from databases, e.g., Patents International (e.g., IMS World Publications). The corresponding content thereof is hereby incorporated by reference.

It will be understood that references to the combination partners (a) and (b) are meant to also include the pharmaceutically acceptable salts. If these combination partners (a) and (b) have, for example, at least one basic center, they can form acid addition salts. Corresponding acid addition salts can also be formed having, if desired, an additionally present basic center. The combination partners having an acid group (for example COOH) can also form salts with bases. The combination partner or a pharmaceutically acceptable salt thereof may also be used in form of a hydrate or include other solvents used for crystallization.

By “combination” according to the invention, there is meant either a fixed combination in one dosage unit form, or a non-fixed combination (or kit of parts) for the combined administration where a compound of the formula (I) and a combination partner (e.g. another active compound or drug) may be administered independently at the same time or separately within time intervals, especially where these time intervals allow that the combination partners show a cooperative, e.g. synergistic effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. a compound of formula (I) and a combination partner, are both administered to a patient simultaneously in the form of a single entity or dosage. The terms “non-fixed combination” or “kit of parts” mean that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

A compound of formula (I) can be administered alone or in combination with one or more other therapeutic compounds or combination partners, possible combination therapy taking the form of fixed combinations or the administration of a compound of the invention and one or more other therapeutic compounds or combination partners being staggered or given independently of one another, or the combined administration of fixed combinations and one or more other therapeutic compounds or combination partners.

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

In a preferred embodiment, the compounds of formula (I) maybe administered to a patient in need thereof at daily dosages of from about 0.03 to about 100.0 mg/kg per body weight, e.g., about 0.03 to about 10.0 mg/kg per body weight. An indicated daily dosage in the larger mammal, e.g., human, is in the range from about 0.5 mg to about 3 mg to about 1.5 g, conveniently administered, for example in divided doses up to four times a day or in retard form. Suitable unit dosage forms for oral administration comprise from about 0.1 to about 500 mg, e.g, 1.0 to about 500 mg active ingredient. For purposes of the present invention, the terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in, a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The pharmaceutical compositions are comprising an amount effective in the treatment of one of the above-mentioned disorders, of a compound of formula (I) or a pharmaceutically acceptable salt thereof together with pharmaceutically acceptable carriers that are suitable for topical, enteral, for example oral or rectal, or parenteral administration and that may be inorganic or organic, solid or liquid. There are pharmaceutical compositions used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting compounds and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilising processes, and comprise approximately from 1% to 99%, especially from approximately 1% to approximately 20%, active ingredient(s).

### BRIEF DESCRIPTION OF THE DRAWINGS

FIG. 1 shows the effects of Compound A on VEGF induced neo-vascularization in vivo. Porous perfluoro-alkoxy-Teflon® chambers containing PBS or VEGF (2 μg/mL) in 0.5 ml of 0.8% w/v agar (containing heparin, 20 U/mL) were implanted subcutaneously in the flank of FVB mice. Animals were treated orally either with Compound A at 12.5, 25 and 50 mg/kg once a day or vehicle (5 ml/kg) starting 4-6 hours before implantation of the chambers and daily for 3 days. VEGF induces the growth of vascularized tissue around the chamber. The angiogenic response can be quantified by measuring the weight and Tie-2 levels of the tissue. Animals were sacrificed 4 days after the implantation. Values are mean±SEM. *p<0.05, statistical significance of inhibition. The total number of animals per group is given in brackets.

FIG. 2 shows the effects of Compound A on the PBS control chamber. Porous perfluoro-alkoxy-Teflon® chambers containing PBS in 0.5 ml of 0.8% w/v agar (containing heparin, 20 U/mL) were implanted subcutaneously in the flank of FVB mice. Animals were treated orally either with Compound A at 50 mg/kg once a day or vehicle (5 ml/kg) starting 4-6 hours before implantation of the chambers and daily for 3 days. Basal tissue formed around the chamber was carefully removed and the weight and Tie-2 levels were quantified. Animals were sacrificed 4 days after the implantation. Values are mean±SEM. *p<0.05, statistical significance of inhibition. The total number of animals per group is given in brackets.

FIG. 3: Effects of Compound A on VEGF induced permeability in vivo. FVB mice pre-treated with Compound A (3, 6.25, 12.5 and 25 mg/kg, p.o., for 5 h) were injected i.v. with Evans blue and challenged 30 minutes later with VEGF injection in the ear. Mice were then sacrificed and VEGF-mediated vessel leakage is quantified by measurement of the area (mm²) of dye that extravasated at the site of VEGF injection using pixel-based threshold in computer-assisted image analysis software. Values are mean±SEM. *p<0.05, statistical significance of inhibition. The total number of animals per group is given in brackets.

FIG. 4: Effects of Compound A on tumor interstitial fluid pressure. (A) Orthotopic BN472 tumor bearing rats containing a pressure sensing catheter inserted in the tumor were treated with Compound A at 25 mg/kg (n=6; initial IFP: 25.4 mmHg±2.8), or with the vehicle control (n=9; 18.2 mmHg±1.5) for 3 consecutive days. The IFP was recorded for 72 h and variations (delta towards initial values) plotted (gray lines: untreated animals; dark line: applied treatment as indicated in the graph). Data presented are means±SEM. (B) Same as in A except that additional animals were treated orally with a lower dose of 12.5 mg/kg Compound A (n=3; initial IFP: 18.8 mmHg±2.3; gray line). Recording was extended for 3 additional days post last treatment to assess recovery patterns. The arrows represent the administration schedule. Gray bars represent night time.

FIG. 5 shows the antitumor effect of Compound A against BN-472 tumor bearing nude rats.

The following Examples illustrate the invention described above; they are not, however, intended to limit the scope of the invention in any way. The efficacy of the compounds of formula (I) and pharmaceutically acceptable salts thereof can also be determined by other test models known as such to the person skilled in the pertinent art.

In the experiments set forth in the following Examples, the animals and compounds were prepared and used/administered as set forth below:

Animals:

Female FVB mice weighing 18 to 20 g were obtained from Charles River Laboratories (les Oncins, France). They were identified via ear markings and kept in groups (6 animals per cage) under normal conditions with free access to food and water and observed daily. Female Brown-Norway (BN) rats were obtained from Harlan Nederland, The Netherlands. They were housed four in a cage with free access to food and water throughout the experiment. Rats were 7-8 weeks of age and weighed between 150 and 170 g when delivered. They were allowed to adapt for 6 days before the experiment was started.

Therapeutic Compound:

Compound A, as free base, was administered at the indicated doses once daily (q.d.) in a vehicle of either 10% NMP/30% PEG300/20% Solutol HS15/40% Water (solution) or 0.5% Methylcellulose suspension (dose volume 5 mUkg) as identified.

Further, in the experiments set forth in the following Examples, statistical comparisons between groups were done with one way ANOVA on Ranks followed by Dunnett's or Dunn's post hoc test. For the chamber model, the Mann-Whitney Rank Sum Test was used. The significant level was set at p<0.05. All statistical calculations were carried out using SigmaStat v 2.03 (Jandel Scientific).

### EXAMPLE 1

### In Vivo Chamber Implant Angiogenesis Assay

Sterile tissue chambers made of perfluoro-alkoxy-Teflon® were filled with 500 μl molten 0.8% w/v agar containing 20 U/mL heparin (Novo Nordisk A/S, Bagsvaerd, Denmark) with or without growth factor (dog VEGF 2 μg/mL or phosphate buffer saline (PBS/O)). The chamber was implanted aseptically under isoflurane anesthesia through a small incision on the back of the animal and the incision was closed by wound clips. Animals were treated orally either with Compound A at 12.5, 25 or 50 mg/kg once a day formulated in a vehicle of NMP/PEG300 (10/90, V/V) or vehicle alone starting 4-6 hours before implantation of the chambers and daily for three days. VEGF induces the growth of vascularized tissue around the chamber. The angiogenic response can be quantified by measuring the weight and Tie-2 levels of the tissue.

Four days after implantation, animals were sacrificed using CO₂. The chambers were recovered from the animal and the vascularized tissue that had formed around each implant was carefully removed and weighed. Tissue samples were homogenized for 30 sec at 24000 rpm (Ultra Turrax T25) after addition of 1 mL of Ripa buffer (Tris-HCl 50 mM, NaCl 120 mM, EDTA 1 mM, EGTA 6 mM, NP-40 1%, Sodium fluoride 20 mM, Pefabloc SC 1 mM, Sodium vanadate 1 mM). The samples were then centrifuged for 1 h at 7000 rpm. The supernatant was filtered using a 0.45 μm syringe filter (Acrodisc® GF, Gelman Sciences, Ann Arbor, Mich., USA) to avoid fat contamination.

Tie-2 is a receptor tyrosine kinase which is specifically expressed on endothelial cells. For Tie-2 level determination, Nunc (Naperville, Ill.) Maxisorp 96-well plates were coated over night at 4° C. with the capture antibody, anti-Tie-2 AB33 (UBI, Hauppauge, N.Y.), with a concentration of 2 μg/mL (100 μL/well). Wells were washed in TPBS (Tween 80 PBS) and blocked by incubating with 3% Top-Block (Juro, Lucerne, Switzerland) for 2 hours at room temperature. 300 μg of protein lysates were added and incubated for 2 hours, and the wells washed three times before addition of a goat anti-mouse Tie-2 antibody (R&D Systems, Minneapolis, Minn.; 0.5 μg/mL) and an alkaline phosphate conjugated anti-goat antibody (Sigma, St. Louis, Mo.; diluted 1:6,000) in TPBS+0.1%Top-Block for 1 hour at room temperature. Tie-2 antibody complexes were detected after incubation with p-nitrophenyl phosphate substrate (Sigma). The absorbance of the spectro-photometric reaction was determined with an ELISA reader at 405 nm. Recombinant human extracellular domain of Tie-2 fused to the constant regions of human IgG1 (sTie-2Fc) dissolved in RIPA buffer was used as standard in a concentration range from 0.1 to 300 ng/well

Four days after the subcutaneous implantation of the VEGF containing chambers, VEGF induced the formation of a thick layer of neovascularised tissue (called capsule) that grew around the implant. PBS containing chambers induced a much thinner layer of tissue with very few blood vessels around control chamber. Tissue formation can be quantified by measuring the weight and the angiogenic response can be quantified by measuring the Tie-2 levels of the capsule. An ELISA assay can quantitatively measure endothelial cells in tissue extracts to evaluate the vascularization of the tissue. The following data summarizing the dose-response effects of Compound A on the angiogenic response induced by VEGF was obtained from the study:

| | % inhibition | | % inhibition | |
|---|---|---|---|---|
| | VEGF-stimulated | | Base line (no GF stimulation) | |
| COMPOUND A (n) | exp. 1 | exp. 2 | exp. 1 | exp. 2 |
| Capsule weight | | | | |
| 12.5 mg/kg p.o. | 64 ± 33 (3) | 74 ± 24 (4) | n.d. | n.d. |
| 25 mg/kg p.o. | 84 ± 5 * (3) | 106 ± 28 * (4) | n.d. | n.d. |
| 50 mg/kg p.o. | 88 ± 12 * (4) | 121 ± 11 * (6) | 14 ± 11 (2) | 12 ± 10 (3) |
| Total Tie-2 | | | | |
| 12.5 mg/kg p.o. | n.d. | 58 ± 22 (4) | n.d. | n.d. |
| 25 mg/kg p.o. | n.d. | 113 ± 38 * (4) | n.d. | n.d. |
| 50 mg/kg p.o. | n.d. | 145 ± 9 * (6) | n.d. | 29 ± 9 * (3) |

Values are mean ± SEM.
* p < 0.05, statistical significance of inhibition, Mann-Whitney Rank Sum Test (n = 2-6 per group per experiment).
n.d.: not determined.

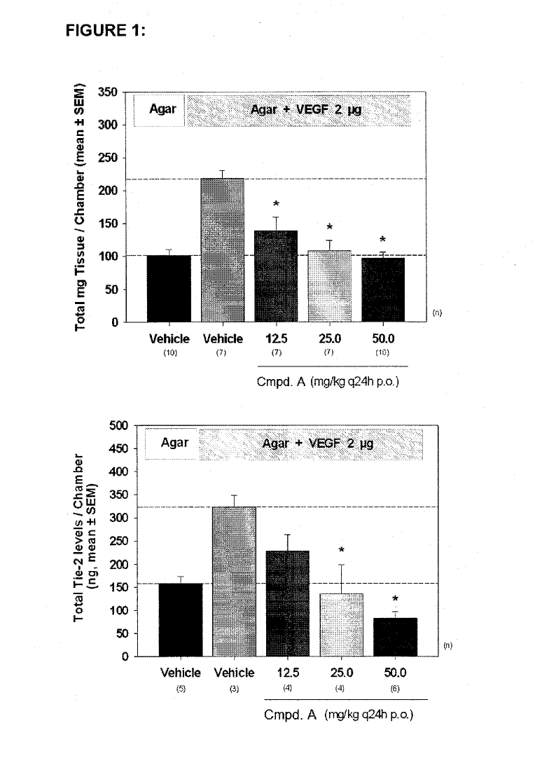

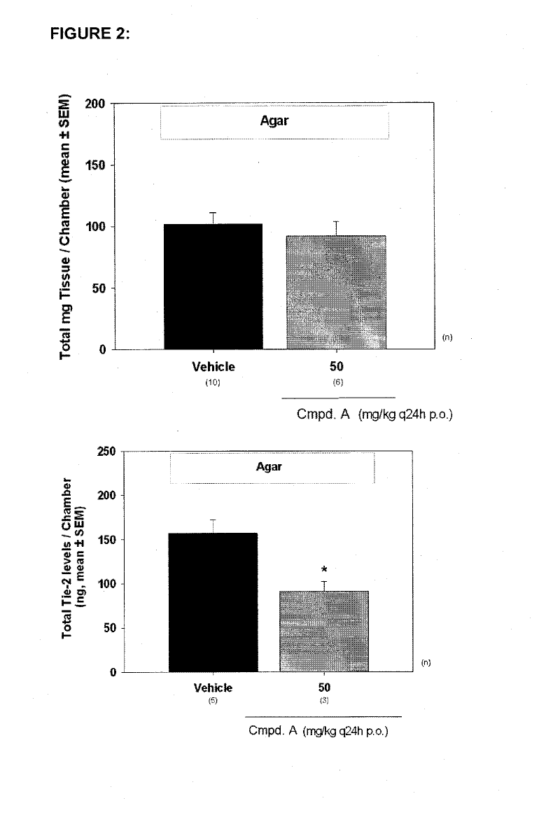

The above data and FIG. 1 show that Compound A, given once a day at 12.5, 25 and 50 mg/kg in solution, inhibited significantly the angiogenic response induced by VEGF as seen by the weight and Tie-2 level of the capsule. Tie-2 levels returned to the baseline or even below with 50 mg/kg given daily. Against basal induced angiogenesis, whereby an agar chamber is implanted without the addition of any growth factor, Compound A at 50 mg/kg did not affect tissue weight (FIG. 2). However, Tie-2 levels were significantly reduced.

### EXAMPLE 2

### In Vivo VEGF Induced Permeability Assay (Miles Assay)

200 μl of Evans blue (0.5%) was injected into the tail vein of female FVB mice. Thirty minutes following administration of the dye, the mice were anaesthesized (3% Isoflurane in O₂, Forene®, Abbott AG, Cham, Switzerland) and then placed on an operating field maintained at a temperature of 39° C. Their ears were extended over a steel cone fitted with a double-sided sticker to expose the dorsal surface. With the aid of a microscope, a 30 G hypodermic needle was then inserted in the skin between the first and second neurovascular bundle of the ear and tunneled for 4-5 mm. Two microliters of VEGF₁₆₄ (10 ng/μl) were injected using a microliter syringe (250 μl, Hamilton, Bonaduz, Switzerland) forming a 2×2 mm sub-dermal blister. Albumin-bounded Evans-blue dye will extravasate at sites of increased microvascular permeability, generating a visible blue spot which provide a measure of vascular permeability. VEGF-mediated vessel leakage is quantified by measurement of the area (mm²) of dye that extravasated at the site of VEGF injection using pixel-based threshold in a computer-assisted image analysis software (KS-400 3.0 imaging system, Zeiss, Germany).

Mice were treated with Compound A for 5 hours before injection of VEGF at doses of 3, 6.25, 12.5 and 25 mg/kg p.o. formulated in a vehicle of 10% NMP/30% PEG300/20% Solutol HS15/40% Water (dose volume 5 mUkg; solution).

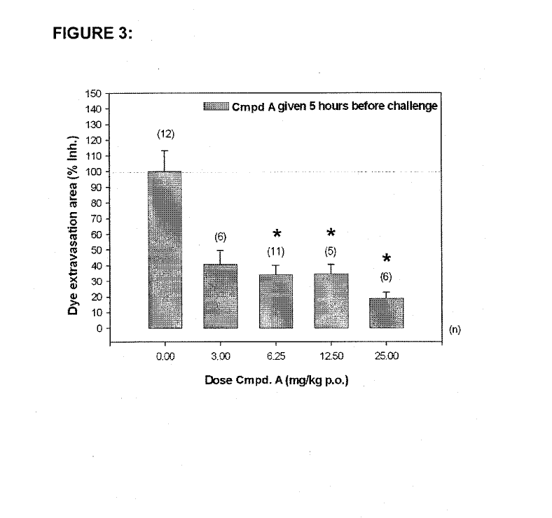

For the foregoing experiment, intradermal VEGF administration in the pinna of the ears produced a marked Evans blue dye extravasation from the local microvasculature. Compound A treatment almost completely abrogated (up to 90%) this effect (FIG. 3). This data demonstrates that Compound A successfully blocks VEGF-induced vessel leakage in vivo.

### EXAMPLE 3

### Efficacy Experiment with Orthotopic BN472 Rat Mammary Carcinoma Model

BN472 tumors were established by orthotopic implantation of tumor fragments into the mammary fat pad of BN rats. Female Brown-Norway (BN) rats (obtained from Charles River (France)) weighing 160-180 g (6-7 weeks of age) were used for all experiments. They were identified by tail markings and kept in groups of 3-5 animals under normal conditions with access to food and water ad libitum. Tumor fragments of 25 mm³ were taken from the cortex region and were transplanted orthotopically under the fat pad of the fourth left mammary gland of the recipients. For efficacy experiments, treatments were always initiated when the mean tumor volume in each group reached 400-500 mm³. Rats were treated with NVP-Compound A at doses of 20 and 40 mg/kg p.o. formulated in 0.5% Methylcellulose (suspension).

The following data summarizing the antitumor activity of Compound A was obtained from the study:

| | Tumor response | | | | Host response | | |
|---|---|---|---|---|---|---|---|
| | | | Δ tumor | Mean fold | Δ Body | % change | |
| | | | volume | change in | weight | of body | Survival |
| | T/C | Regr. | (mean mm³ ± | tumor | (mean g ± | weight | (Survivors/ |
| Treatment | (%) | (%) | SEM) | growth | SEM) | corrected | total) |
| 0.5% MC, 5 ml/kg | 100 | | 4650 ± 686 | 10.9 ± 0.4 | 3.6 ± 1.0 | −0.5 ± 0.6 | 5/5 |
| po q24 h | | | | | | | |
| Compound A | 36 * | — | 1725 ± 283 * | 4.6 ± 0.8 * | 4.0 ± 1.1 | 1.2 ± 0.5 | 5/5 |
| 20 mg/kg, qd, p.o. | | | | | | | |
| Compound A | 23 * | — | 1108 ± 198 * | 3.3 ± 0.2 * | −2.5 ± 0.9 * | −1.8 ± 0.5 | 5/5 |
| 40 mg/kg, qd, p.o. | | | | | | | |

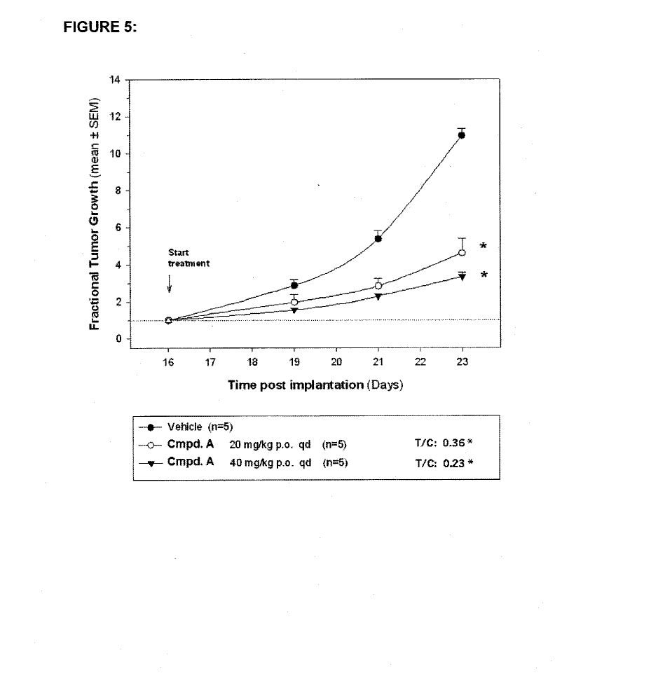

The growth of the primary tumor was significantly (p<0.05) inhibited by treatment with 20 mg/kg/day (T/C 36%) or 40 mg/kg/day (T/C 23%). (FIG. 5) Treatment was well tolerated with no significant body weight loss at any tested dose.

### EXAMPLE 4

### Tumor Interstitial Fluid Pressure

The IFP of BN472 tumors was measured in conscious, freely moving rats maintained in their home cage using an adapted fully implantable miniaturized radio-telemetry system (Data Sciences Int., St. Paul, Minn.) composed of 4 basic components: an implantable transmitter (AM unit, model TLM-PAC10, volume: 1.1 cc, weight: 1.4 g) which continuously senses and transmits information from within the animal, one receiver located under the home cage, a matrix interface for coordination of signals and a computer-based data acquisition system for collection, analysis and storage of data. The body of the transmitter was implanted s.c. under aseptic conditions into the flank of the animal under isofluorane anaesthesia (3% Isoflurane in O2). Briefly, once animals are fully unconscious, they are placed on a heated blanket and the abdominal area is shaved. The ventral surface of the abdomen is then prepared by swabbing the skin with povidone iodine surgical scrub. With the animal supine, a skin incision of approximately 30 mm is made along the midline of the abdomen and the skin separated from the muscle wall building an air pocket. The sterile transmitter is then positioned in this pocket and the sensing catheter tunneled subcutaneously towards the tumor site (lowest mammary fat pad). The sensing pressure catheter was inserted into the body of the tumor (4-5 mm depth) and secured at the site of entry with tissue adhesive (Vetbond; 3M Company). Fluid communication between the tip of the pressure catheter and the tumor was tested by gentle compressing and decompressing the tubing connected to the telemetry transducer using a clamp. The catheter implantation was quoted as good if the readings before and after this test did not differed by more than 1 mm Hg. However, in most of the implanted tumors, intra-tumoral IFP pulse pressure waveform curves could be recorded unstintingly with high resolution confirming even more strongly the adequate sensing catheter placement. The total operation time for the implantation of the telemetry transmitter was about 20 min. Postoperative analgesia was provided using Buprenorphin (Temgesic®, Reckitt and Colman) injection of 0.05 mg/kg s.c. twice, immediately after surgery and 8-12 h later. Following surgery, the unconscious animal is placed on soft material in a clean cage with water ad libitum. An external heat lamp is used to maintain body temperature. The animals were allowed a period of 2 days to recover from surgery before starting acquisition of any physiological data. IFP was recorded continuously in all animals over 24 hours up to 5 days, and analyzed in 1-min cyclic runs for 10 sec, with a 500-Hz sampling rate. Values presented are means over 15 min periods. Rats were treated with Compound A at doses of 20 and 40 mg/kg p.o. formulated in 0.5% Methylcellulose (suspension).

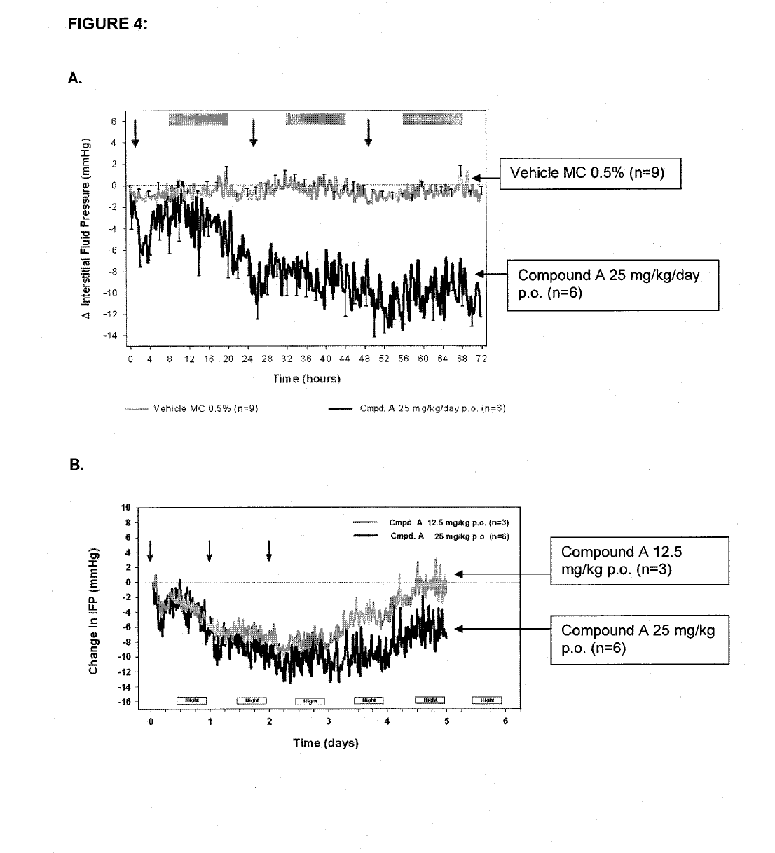

At the highest dose of Compound A tested (25 mg/kg), a clear and significant reduction in IFP was already detected 4 hours post treatment (−25% at max, 22 hours post dosage) (FIG. 4A). A second treatment of Compound A at a dose of 25 mg/kg (at time point 24 hours) resulted in sustained and superior IFP inhibition (−36%) in comparison to the single dose study. A third treatment resulted in a small but sustained additional IFP inhibition (−43%) for 48 hours. Thus, at this dose level in this tumor model, maximum IFP level inhibition by Compound A given at tolerated dosage regimen was likely reached. At a dose of 12.5 mg/kg, a very similar pattern was obtained over the whole three days of treatment with the IFP returned to initial values by 60 hours post last treatment (B). By contrast, the IFP diminution obtained for the 25 mg/kg dose level group did not normalize even 72 h post last treatment

1-11. (canceled)

12. A method of treating a VEGF-driven angiogenic disease comprising administering a therapeutically effective amount of a compound of formula (I), 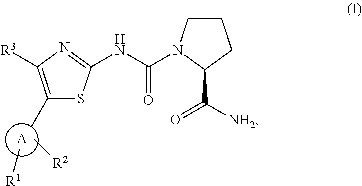
or a pharmaceutically acceptable salt thereof, wherein
A represents a heteroaryl selected from the group consisting of: 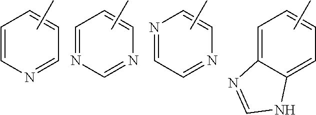
R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkyl, C₁-C₇-alkylamino, di(C₁-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl,
wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide).

13. The method of claim 12, where the compound of the formula (I) is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof.

14. The method of claim 12, wherein the VEGF-driven angiogenic disease to be treated is Rheumatoid arthritis, Synovitis, Bone and cartilage destruction, Osteomyelitis, Pannus Growth, Osteophyte formation, Hepatitis, Pneumonia, Glomerulonephritis, Asthma, Nasal polyps, Transplantation, Liver generation, Retinopathy of prematurity, Age macular degeneration, Diabetic retinopathy, Chroidal and other intraocular disorders, Leukomalacia, Thyroiditis, Thyroid enlargement, Lympopholiferative disorders, Karposi's sarcoma, Haematologic malignacies (e.g., haemangiomas), Obesity, Spinal cord injury, Acutemyocardial infarction, Pulmonary, cerebral and retinal oedema, or further any combinations thereof.

15. The method of claim 12, wherein the VEGR-driven angiogenic disease has acquired resistance to agents that target VEGF and/or VEGFR family members.

16. The method of claim to claim 15, wherein the disease to be treated is a disease is Rheumatoid arthritis, Synovitis, Bone and cartilage destruction, Osteomyelitis, Pannus Growth, Osteophyte formation, Hepatitis, Pneumonia, Glomerulonephritis, Asthma, Nasal polyps, Transplantation, Liver generation, Retinopathy of prematurity, Age macular degeneration, Diabetic retinopathy, Chroidal and other intraocular disorders, Leukomalacia, Thyroiditis, Thyroid enlargement, Lympopholiferative disorders, Karposi's sarcoma, Haematologic malignacies (e.g., haemangiomas), Obesity, Spinal cord injury, Acutemyocardial infarction, Pulmonary, cerebral and retinal oedema, or further any combinations thereof.

17. The method of claim 13, wherein the VEGF-driven angiogenic disease to be treated is Rheumatoid arthritis, Synovitis, Bone and cartilage destruction, Osteomyelitis, Pannus Growth, Osteophyte formation, Hepatitis, Pneumonia, Glomerulonephritis, Asthma, Nasal polyps, Transplantation, Liver generation, Retinopathy of prematurity, Age macular degeneration, Diabetic retinopathy, Chroidal and other intraocular disorders, Leukomalacia, Thyroiditis, Thyroid enlargement, Lympopholiferative disorders, Karposi's sarcoma, Haematologic malignacies (e.g., haemangiomas), Obesity, Spinal cord injury, Acutemyocardial infarction, Pulmonary, cerebral and retinal oedema, or further any combinations thereof.

18. The method of claim 13, wherein the VEGR-driven angiogenic disease has acquired resistance to agents that target VEGF and/or VEGFR family members.

19. The method of claim to claim 18, wherein the disease to be treated is a disease is Rheumatoid arthritis, Synovitis, Bone and cartilage destruction, Osteomyelitis, Pannus Growth, Osteophyte formation, Hepatitis, Pneumonia, Glomerulonephritis, Asthma, Nasal polyps, Transplantation, Liver generation, Retinopathy of prematurity, Age macular degeneration, Diabetic retinopathy, Chroidal and other intraocular disorders, Leukomalacia, Thyroiditis, Thyroid enlargement, Lympopholiferative disorders, Karposi's sarcoma, Haematologic malignacies (e.g., haemangiomas), Obesity, Spinal cord injury, Acutemyocardial infarction, Pulmonary, cerebral and retinal oedema, or further any combinations thereof.

20. A method of treating a VEGF-driven angiogenic disease comprising administering a therapeutically effective amount of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) in combination with at least one VEGF or VEGFR targeting agent selected from the group consisting of Bevacizumab, anti-VEGF, Ranibizumab AVE0005, anti-VEGF HuMV833, anti-VEGF 2C3, anti-VEGF CBO-P11, Sutent, Sorafenib, Vatalanib, Zactima, Midostaurin, Angiozyme, AG-013736, Lestautinib, CP-547,632, CEP-7055, KRN633, NVP-AEE788, IMC-1211, ZK260253, Semaxanib, E-7107, AS-3, Cand5 and PTC-299; and the HSP90 inhibitors CNF1010, CNF2024, tanespimycinm, alvespimycin, IPI504, SNX5422 and NVP-AUY922,
wherein the active ingredients are present in each case in free form or in the form of a pharmaceutically acceptable salt, and optionally at least one pharmaceutically acceptable carrier, for simultaneous, separate or sequential use.
